(19) United States
(12) Patent Application Publication
(10) Pub. No.: US 2026/0076885 A1
(43) Pub. Date: Mar. 19, 2026

(54) Complexation of Oxybenzone with C-methylresorcin[4]arene

(71) Applicant: University of Cincinnati, Cincinnati, OH (US)

(72) Inventors: Harshita Kumari, Mason, OH (US); Xu Kang, Cincinnati, OH (US)

(73) Assignee: University of Cincinnati, Cincinnati, OH (US)

(21) Appl. No.: 19/402,209

(22) Filed: Nov. 26, 2025

Related U.S. Application Data
(62) Division of application No. 17/838,754, filed on Jun. 13, 2022, now abandoned.
(60) Provisional application No. 63/209,466, filed on Jun. 11, 2021.

(51) Int. Cl. A61K 8/35; A61K 8/34; A61Q 17/04

(52) U.S. Cl. CPC A61K 8/35; A61K 8/347; A61Q 17/04

(57) ABSTRACT

The present invention involves the formation of host-guest complexes between C-methylresorcin[4]arene and Oxybenzone (OXB). NMR spectroscopy confirmed the formation of a weak host-guest complex and molecular dynamics docking simulations indicated that this complex likely had a 1:1 stoichiometry. Furthermore, skin permeation testing revealed that complexation by C-methylresorcin[4]arene significantly reduced the amount of OXB that permeated skin. These results show the potential of supramolecular complexation for improving the stability and decreasing the skin permeability of OXB, thus limiting harmful side effects.

### CROSS REFERENCE TO RELATED APPLICATIONS

This application is a continuation of U.S. application Ser. No. 17/838,754 filed Jun. 13, 2022, which claims benefit of U.S. Provisional Application Ser. No. 63/209,466, filed Jun. 11, 2021, which application is hereby incorporated by reference in its entirety.

### TECHNICAL FIELD

The present invention relates to complexes of oxybenzone.

### BACKGROUND OF THE INVENTION

Chronic exposure to UV radiation can cause various skin problems including sunburn, aging, and even melanoma (skin cancer). Mid-range ultraviolet light (UVB, 280-320 nm) has been found to cause direct DNA damage; however, wavelengths that are not directly absorbed by DNA (UVA, >320 nm) can also induce skin damage. In particular, DNA damage can be induced by wavelengths from 312 nm (in the UVB region) up to 434 nm (in the near-visible region) via the production of cyclobutane pyrimidine dimers and other photoproducts, which are associated with mutagenesis and cancer. This damage is not caused by the direct absorption of light but by the activation of photosensitizers that can generate singlet oxygen species. By absorbing or reflecting UV radiation, sunscreens can prevent oxidative damage to DNA, thus keeping our skin from aging and protecting us from skin cancer.

However, the instability and skin permeation of sunscreen ingredients remain concerning to both the industry and consumers. Oxybenzone (OXB) is a commonly used ingredient in sunscreens (FIG. 1A) that provides effective broad-spectrum ultraviolet coverage. However, beginning Jan. 1, 2021, Hawaii has banned OXB-containing sunscreens to preserve coral reefs.

Moreover, OXB may have keratinocyte toxicity, interrupt human hormones, and penetrate skin to enter the blood circulation system. OXB has good permeability through the blood-brain barrier (BBB) and can significantly affect the neural development of children. Unfortunately, OXB can also be found in human breast milk, thus endangering children's health. In vivo studies using the Franz diffusion cell have shown that 10% of OXB can penetrate human skin, with 0.4% entering the bloodstream and being eliminated through urine. This result indicates that OXB can form complexes with substances in the adult human body to improve its water solubility, thus allowing urine excretion; however, little is known about the efficiency of this process in children. A direct association has recently been found between OXB and Hirschsprung's disease (a neonatal intestinal abnormality derived from the failed migration of enteric neural crest cells) in infants under normal use conditions of sunscreen products by expecting mothers. Although OXB permeation is not known to present any acute danger to adults, chronic effects and pediatric toxicities are still not fully understood. Furthermore, although OXB has recently been shown to be relatively stable as compared to avobenzone (AVOB) and ecamsule (ECAM), it can be easily oxidized under natural sunlight. As the oxidation of OXB can inactivate antioxidant systems, OXB as a sunscreen ingredient is a potential source of skin irritation. Thus, both the instability and skin permeability of OXB are growing concerns.

### SUMMARY OF THE INVENTION

In one embodiment, the present invention addresses that need with a composition comprising a cocrystal complex of oxybenzone with C-methylresorcin[4]arene (RsC1). In another embodiment, the present invention involves a sunscreen formulation comprising a cocrystal complex of oxybenzone with C-methylresorcin[4]arene (RsC1). In one embodiment, the sunscreen formulation includes from about 2 to about 10% by weight of the cocrystal complex. In one embodiment, the sunscreen formulation includes from about 4 to about 8% by weight of the cocrystal complex.

In another embodiment, the sunscreen formulation further includes a cosmetically acceptable carrier. In one embodiment, the carrier includes one or more carriers selected from the group consisting of preservatives, emollients, emulsifying agents, surfactants, moisturizers, gelling agents, thickening agents, conditioning agents, film-forming agents, stabilizing agents, anti-oxidants, texturizing agents, gloss agents, mattifying agents, solubilizers, pigments, dyes, and fragrances.

In another embodiment of the present invention, a method of protecting a subject's skin from sun damage is provided. The method involves applying a therapeutically effective amount of a cocrystal complex of oxybenzone with C-methylresorcin[4]arene (RsC1) to the skin of the subject. In one embodiment, the method involves applying a therapeutically effective amount of a sunscreen formulation including a cocrystal complex of oxybenzone with C-methylresorcin[4]arene (RsC1) and one or more carriers to the skin of the subject.

### BRIEF DESCRIPTION OF THE DRAWINGS

The foregoing summary, as well as the following detailed description of preferred embodiments of the application, will be better understood when read in conjunction with the appended drawings.

FIG. 1A is an image showing the molecular structure of OXB.

FIG. 1B is an image showing the molecular structure of RsC1.

FIG. 2 is a graph showing 1H NMR titration of OXB with RsC1 in ethanol-d1.

FIG. 3 is a DOSY spectra of (a) OXB, (b) RsC1, and (c) a 1:4 mixture of OXB+RsC1 in ethanol-d1.

FIG. 4 is an image showing possible conformation of RsC1-OXB host-guest complexes in ethanol-d1.

FIG. 5 is an image showing spatial distribution functions of OXB (green) and solvent (blue) associated with a RsC1 molecule.

FIG. 6 is a pair of graphs showing the encapsulation geometry of RsC1-OXB defined by host-host distance (left) and host-guest-host angle (right).

FIG. 7 is a graph showing the flexibility of the top (orange) and bottom (blue) openings of RsC1.

FIG. 8 is a graph showing the distributions of average number of RsC1 molecules associated with an OXB molecule at cutoff radii of 2-15 Å.

FIG. 9 is a graph showing skin permeation fractions of the OXB group (20.1%±4.7%; mean±SD, n=3) and the OXB+RsC1 group (11.1%±3.2%; mean±SD, n=3).

### DETAILED DESCRIPTION

One skilled in the art will recognize that the various embodiments may be practiced without one or more of the specific details described herein, or with other replacement and/or additional methods, materials, or components. In other instances, well-known structures, materials, or operations are not shown or described in detail herein to avoid obscuring aspects of various embodiments of the invention. Similarly, for purposes of explanation, specific numbers, materials, and configurations are set forth herein in order to provide a thorough understanding of the invention. Furthermore, it is understood that the various embodiments shown in the figures are illustrative representations and are not necessarily drawn to scale.

Reference throughout this specification to “one embodiment” or “an embodiment” means that a particular feature, structure, material, or characteristic described in connection with the embodiment is included in at least one embodiment of the invention, but does not denote that they are present in every embodiment. Thus, the appearances of the phrases “in an embodiment” or “in another embodiment” in various places throughout this specification are not necessarily referring to the same embodiment of the invention. Further, “a component” may be representative of one or more components and, thus, may be used herein to mean “at least one.”

The term “therapeutically effective amount” means an amount of a compound according to the disclosure which, when administered to a patient in need thereof, is sufficient to effect treatment for disease-states, conditions, or disorders for which the compounds have utility. Such an amount would be sufficient to elicit the biological or medical response of a tissue, system, or patient that is sought by a researcher or clinician. The amount of a compound of according to the disclosure which constitutes a therapeutically effective amount will vary depending on such factors as the compound and its biological activity, the composition used for administration, the time of administration, the route of administration, the rate of excretion of the compound, the duration of treatment, the type of disease-state or disorder being treated and its severity, drugs used in combination with or coincidentally with the compounds of the disclosure, and the age, body weight, general health, sex, and diet of the patient. Such a therapeutically effective amount can be determined routinely by one of ordinary skill in the art having regard to their own knowledge, the prior art, and this disclosure.

Various attempts have been made in recent years to reduce the side effects of OXB. Some research proposed increasing the viscosity of sunscreen formulations to reduce skin permeation by OXB. Although large amounts of a thickening agent were found to decrease skin permeation, this effect was offset when the solution was applied as a thin layer. Other studies used hydroxypropyl-β-cyclodextrin to decrease OXB skin permeation and accumulation. Increases in permeation and accumulation were observed with 10% (w/w) hydroxypropyl-β-cyclodextrin, whereas both decreased with 20% (w/w) hydroxypropyl-β-cyclodextrin. These data indicated the potential formation of a drug reservoir on the skin, but complex formation was not verified using NMR nor other analytical techniques. Moreover, no studies have focused on using macrocycles to modify OXB and potentially address drug safety issues.

The confined space of a host molecule can provide both a boundary and free space within the cavity for a guest. A host can also provide conformation selectivity, depending on the free space available, the flexibility of the host, and the interactions between the host and the guest. Previous studies have shown that the hydrophobic cavities of many macrocycles can fine-tune the chemical properties of guests by, for example, improving solubility and stability, providing product selectivity, and changing fluorescence properties.

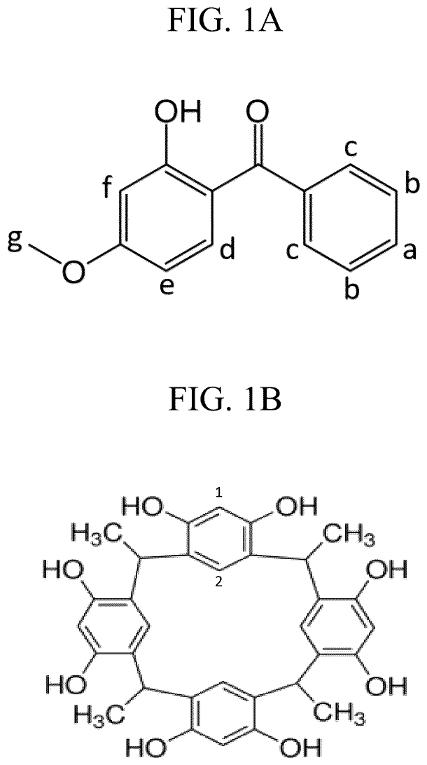

The present invention has developed a novel complex using the effect of host-guest complexation with C-methylresorcin[4]arene (RsC1) (FIG. 1B), a macrocycle host, on the chemical properties of OXB (FIG. 1A). Resorcinol[4]arenes represent a class of cyclic polyphenolic compounds obtained from the condensation reaction of resorcinol with several aldehydes in acidic solutions. Interestingly, the flexibility in changes of electron-rich upper-rim bunches and lower-rim alkyl chains with distinctive substituents driven to a wide assortment of tunable host molecules. Amidst cyclic polyphenolic cavitands, resorcinarenes have broadly been inspected in host-guest chemistry due to their conical shape to develop valuable (bio)materials and sensors. A variety of guests, from cationic to neutral molecules, have been found to embed into this cavity, through C—H . . . π, cation . . . π and π . . . π interactions. This ability to act as a host, together with its adaptability and affinity towards hydrogen bonding, makes resorcinol[4]arenes a perfect candidate for cocrystallizations. Despite enjoying omnipresent investigation in chemical studies, cyclic polyphenol-based host-guest chemistry including pharmaceutical actives is undoubtedly in its earliest stages. The chemical structure of C-methylresorcin[4]arene (RsC1) is shown in FIG. 1B. Complex formation was examined using NMR spectroscopy, the inclusion geometry was elucidated using molecular dynamics docking simulations, and the effects of host-guest complexation on skin permeation were determined using the Franz diffusion cell.

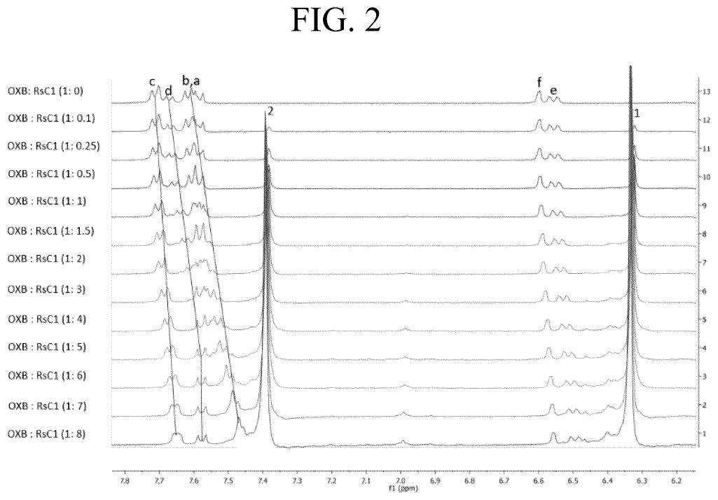

Host-guest complexation between RsC1 and OXB was shown using 1H NMR spectroscopy. FIG. 2 shows the 1H NMR spectra of OXB with increasing concentrations of RsC1. As RsC1 was added, the aromatic peaks at 7.0-7.4 ppm shifted upfield, indicating formation of a complex between the RsC1 and OXB. The peaks continued to move up to an OXB/RsC1 ratio of 1:8, however, indicating that under these conditions the OXB-RsC1 interaction was too weak to achieve complete saturation.

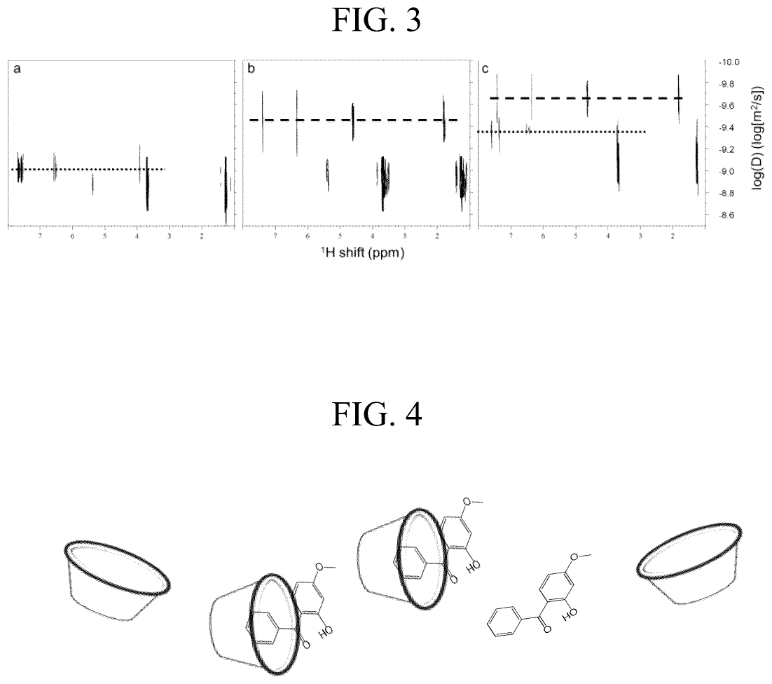

Complexation between OXB and RsC1 can be shown using 2D 1H diffusion-ordered spectroscopy (DOSY). The DOSY spectra of OXB, RsC1, and the OXBt+RsC1 complex are shown in FIG. 3. The DOSY spectra of pure OXB and RsC1 gave diffusion rates of approximately 10-9 and 10-9.4 m2/s, respectively (FIGS. 3A and 3B). In contrast, the DOSY spectrum of the OXB+RsC1 mixture (60 mg/ml+143 mg/ml) gave slower diffusion rates of 10-9.4 and 10-9.7 m2/s corresponding to OXB and RsC1, respectively (FIG. 3C). This can be taken as evidence of binding, but not complete saturation of OXB by RsC1 In that case, the OXB peaks would be expected to correspond to a full-formed complex and yield an equivalent or slower diffusion rate than the RsC1 peaks.

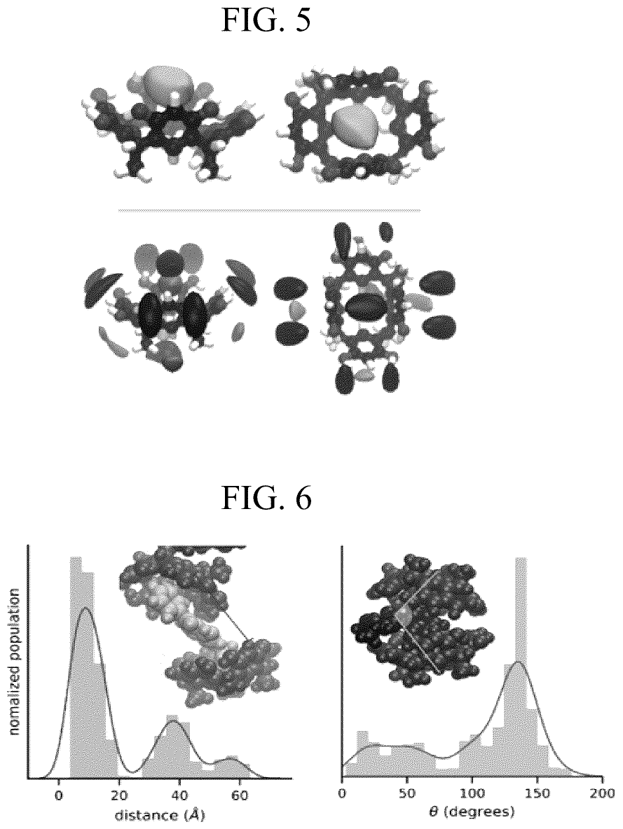

FIG. 4 shows a possible conformation for the OXB-RsC1 complex, in which the benzene ring of OXB is associated with the upper rim of a RsC1 molecule, whereas the —CH3 group of OXB interacts with the lower rim of another RsC1 molecule. This conformation may explain why the complexation ratio could not be determined. OXB is predicted to primarily interact with the inner cavity of the host molecule, as indicated by the high-density areas in the calculated spatial distribution function of OXB (FIG. 5).

The nature of the RsC1-OX3 interaction was further characterized by monitoring the distance and angle between two encapsulating RsC1 molecules during interaction with an OXB molecule. The distance between two RsC1 molecules interacting through an OXB molecule was found to be centered at 10 Å (FIG. 6, left). In the distribution, two other peaks were observed (centered at 39 and 58 Å), corresponding to macrocycle pairs that had dissociated completely. Furthermore, the most favorable angle of interaction was found to be near 150°. These simulation results indicate that the partial encapsulation of an OXB molecule by two RsC1 molecules is possible.

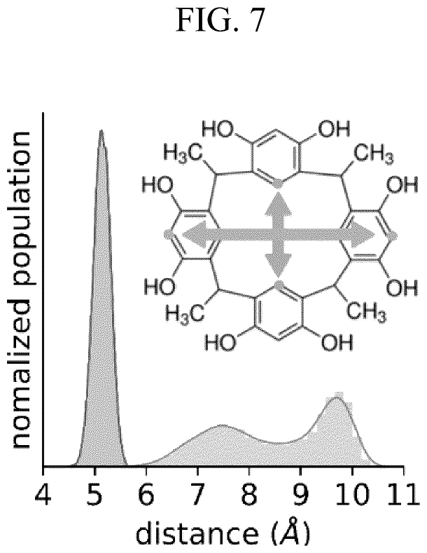

To quantify the free space available in the RsC1 molecule, the size of the openings at the top and bottom of the macrocycle were monitored during the simulations (FIG. 7). Using these measurements, the size of molecules that can associate with and sit within RsC1 can be estimated. The bottom opening showed narrow fluctuations around 5 Å, whereas the top opening showed two ranges centered at 7.5 and 9.75 Å. The bimodal nature of this distribution suggests that when associated with OXB, the top of the macrocycle may stretch to accommodate the guest.

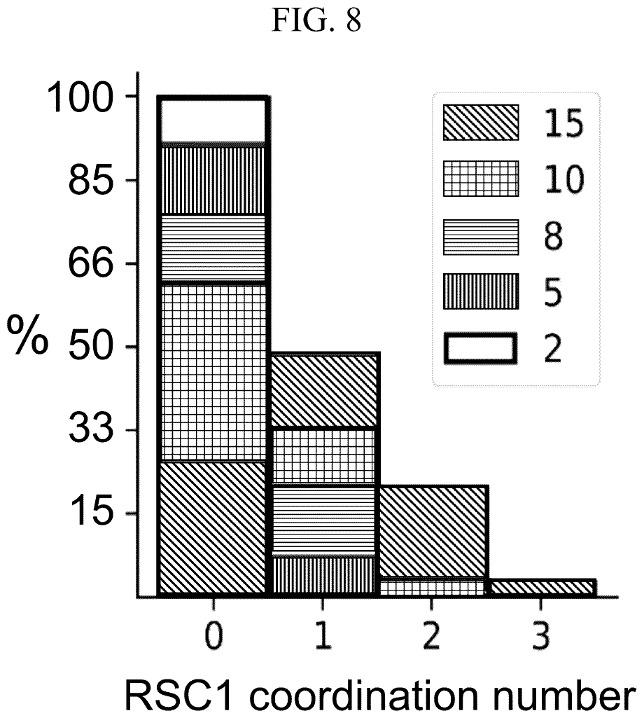

By tracking the association of RSC1 and OXB during the molecular dynamics simulations, the average number of hosts associated with a guest molecule were quantified (FIG. 8). These results are based on the population of RSC1 molecules observed near the OXB molecule within a preset cutoff radius. Increasing the cutoff radius increases the probability of detecting RsC1 molecules that interact with the OXB molecule; however, larger cutoff radii, may also include RsC1 molecules that do not interact with the OXB molecule or that interact with the backside of another RsC1 molecule. Therefore, the 8 Å and 10 Å cutoff radii were determined to be the most relevant (FIG. 8). The most commonly observed association structure during the simulations involved a 1:1 interaction between RsC1 and OXB, which occurred approximately 20% and 33% of the time at 8 and 10 Å, respectively. In these cutoff ranges, 2:1 interactions between RsC1 and OXB were also observed, but the probability was nearly 10 times lower than that of the 1:1 interaction. These simulation results, which are in agreement with the experimental findings, show that the most common interaction between RsC1 and OXB is a 1:1 host-guest complex, with the OXB guest sitting deep inside the RsC1 host. The interaction of two RsC1 with one OXB can also occur in this system, with the guest located between the two hosts.

The complexes of the present invention may be used for a variety of cosmetic uses, including incorporating them into sunscreen formulations that provide protection of the skin from UV radiation and visible light radiation. Such compositions will include a UV-absorbing agent and, optionally, a visible light absorbing inorganic pigment particle in the composition. The ingredients are combined with a “cosmetically-acceptable topical carrier,” i.e., a carrier for topical use that is capable of having the other ingredients dispersed or dissolved therein, and possessing acceptable properties rendering it safe to use topically. The carrier may include one or more carriers selected from the group consisting of preservatives, emollients, emulsifying agents, surfactants, moisturizers, gelling agents, thickening agents, conditioning agents, film-forming agents, stabilizing agents, anti-oxidants, texturizing agents, gloss agents, mattifying agents, solubilizers, pigments, dyes, and fragrances.

These compositions may be made into a wide variety of delivery forms. These forms include, but are not limited to, suspensions, dispersions, solutions, or coatings on water soluble or water-insoluble substrates (e.g., substrates such as organic or inorganic powders, fibers, or films). Suitable product forms include lotions, creams, gels, sticks, sprays, ointments, mousses, and compacts/powders. The composition may be employed for various end-uses, such as recreation or daily-use sunscreens, moisturizers, cosmetics/make-up, cleansers/toners, anti-aging products, or combinations thereof. The compositions of the present invention may be prepared using methodology that is well known by an artisan of ordinary skill in the field of cosmetics formulation.

The complexes of the present invention may be included in formulations at various concentrations, depending on the specific end use and carrier. In one embodiment, the complexes comprise from about 2 to about 10 wt % of a cosmetic formulation. In another embodiment, the complexes comprise from about 4 to about 8 wt % of a cosmetic formulation.

### EXAMPLES

### Example 1: Skin Permeation

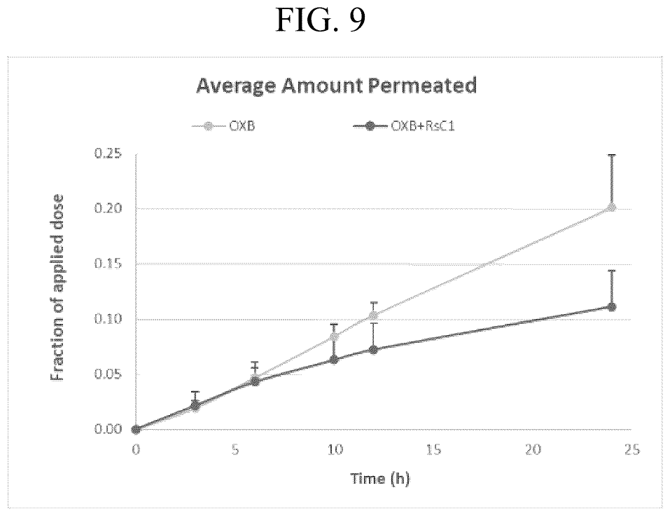

The skin permeation of OXB was evaluated using 6% OXB formulations with and without RsC1 in a Franz diffusion cell (FIG. 9 and Table 1). In the OXB only group, 20.1% of the applied dose was found to penetrate the skin after 24 h. In the OXB+RsC1 group, skin permeation was significantly reduced and only 11.1% of the applied dose penetrated the skin (Student's unpaired t-test, p=0.016). This result demonstrates that RsC1 complexation has an effect on OXB skin permeation.

The amounts of OXB remaining on the skin surface, inside the skin, and on the surface of the donor chamber cap were also determined at the end of the skin permeation experiments. The average total recovery of OXB from the permeation study was approximately 75%. The recovery data indicate that a significant amount of OXB remained on the skin surface and that the amount of OB remaining inside the skin was low when OXB was complexed with RsC1.

In addition, the amount of RsC1 that permeated through the skin to the receptor chamber was determined using an HPLC assay. As the amount of RsC1 permeated through the skin was found to be negligible, the skin permeation of RsC1 was insignificant under the conditions examined in this study. Together, these results suggest that RsC1 complexes OXB without permeating the skin, which could improve the safety of OXB-based sunscreen products.

TABLE 1
Recovery of OXB during skin permeation tests

| | OXB | | OXB + RsC1 | |
|---|---|---|---|---|
| | Average (%) | SD | Average (%) | SD |
| Receptor chamber | 20.1 | 4.7 | 11.1 | 3.2 |
| Rinse 1 | 24.0 | 7.5 | 5.8 | 0.6 |
| Rinse 2 | 22.9 | 5.8 | 61.6 | 10.6 |
| Skin cut-up | 2.7 | 2.2 | 0.3 | 0.1 |
| Donor chamber cap | 0.6 | 0.4 | 0.9 | 0.2 |
| Total OXB recovered | 70.3 | 9.2 | 79.7 | 8.0 |

Table 1 shows the recovery of OXB during skin permeation tests. Rinse 1: rinsing once using receptor chamber solution (PBS with 0.02% sodium azide and 3% cyclodextrin); Rinse 2: rinsing four times using a methanol/water (85:15) solution. The values for the rinses, cut-up skin, and donor chamber cap were adjusted to correct for the extraction method and RsC1 interference during the OXB assay.

### Example 2: Materials

OXB and β-cyclodextrin were purchased from Sigma-Aldrich. RsC1 was synthesized using a solvent-free protocol as described by Tunstad et al. and Elidrisi et al. Phosphate-buffered saline (PBS) tablets (pH 7.4) were purchased from MP Biomedicals (Solon, OH). Sodium azide was purchased from Acros Organics (Morris Plains, NJ). Deuterated solvents were purchased from Sigma-Aldrich and Cambridge Isotope Laboratories. Split-thickness human cadaver skin (from males and females between the ages of 45 and 70 years) was obtained from the New York Firefighters Skin Bank (New York, NY). TEWL was measured using Delfin vapometer (Kuopio, Finland). NMR spectra were obtained using an AV 400 MHz NMR spectrometer (Bruker AV-400, Coventry, UK).

### Example 3: NMR Titration

Stock solutions of OXB and RsC1 were prepared using ethanol-d1. In different NMR tubes, the host (RsC1) was gradually added to the guest (OXB) at molar ratios from 0:1 to 8:1, ensuring that each tube contained the same amount of deuterated solvent (0.8 mL). The tubes were sonicated and shaken vigorously until the solutions were well mixed, and then NMR spectra were recorded.

### Example 4: DOSY

Very concentrated OXB and OXB+RsC1 solutions were prepared in ethanol-d1 (60 mg/ml OXB). The host (RsC1) was added to the guest (OXB) at a molar ratio of 1:1. The tubes were sonicated and shaken vigorously until the solutions were well mixed, and then DOSY spectra were collected.

### Example 5: Docking

Host-guest docking was explored using classical molecular dynamics simulations. Systems with host/guest ratios of 1:1, 2:1, and 4:1 were simulated over long timescales to sample the docking interactions. The molecular dynamics simulations were conducted using the Gromacs-2019.2 suite of programs, utilizing the CHARMM general force field to represent the host and guest molecules. The presented results were sampled from production simulations of 200 ns, which were preceded by system equilibration using the Nose-Hoover thermostat (0.1 ps coupling time) and the Parrinello-Rahman barostat (1.0 ps coupling time). Spatial distribution functions were calculated using the trajectory analyzer and visualizer (TRAVIS).

### Example 6: Skin Preparation

Skin permeation studies of OXB were performed using split-thickness human skin in a Franz diffusion cell with a 0.7 cm2 diffusion area. The skin samples were labeled with the code of the skin donor (n=3, CC, RT, or SL) and the same group of skin donors was used in each experiment. To prepare the skin samples, the cadaver skin was cut into 1.5 cm×1.5 cm pieces, thawed in Petri dishes with PBS at room temperature for 2 h, and then patted dry with Kimwipes before being mounted onto the diffusion cell with the stratum corneum side facing up. PBS containing 0.02% sodium azide as a preservative and 3% cyclodextrin as a solubilizing agent for OXB was used as the solution in the receptor chamber. The TEWL of the skin sample was measured to ensure skin integrity before the permeation study. For TEWL measurements, a vapometer was placed on top of the donor chamber of the diffusion cell and the measurement was repeated until a constant TEWL value was obtained (2-3 measurements, 15 min apart). Only skin samples with TEWL values of <10 g/m2/h were used for permeation experiments.

### Example 7: Permeation Study

After the TEWL measurement, the skin in the diffusion cell was allowed to equilibrate for 1 h. During this equilibration period, the skin surface was maintained at 32±1° C. The receptor medium was stirred with a magnetic stirrer at 600 rpm to ensure uniform mixing. The permeation study was initiated by applying 2 mg/cm2 of the test formulation on the skin surface in the diffusion cell. The dose of 2 mg/cm2 was selected based on FDA guidelines. The test formulations were 6% OXB and 6% OXB+RsC1 (1:1) in ethanol. At 0, 3, 6, 10, 12, and 24 h after dosing, 0.5 mL samples were withdrawn from the receptor chamber and immediately replaced with the same volume of fresh PBS solution to maintain a constant receptor solution volume. At the end of the experiment, the residual formulation in the donor compartment was recovered by rinsing once with the receptor chamber solution (PBS with 0.02% sodium azide and 3% cyclodextrin) and four times with methanol/water (85:15) solution (each time with 0.5 ml for 30 s). The treated areas were then dried with Kimwipes. The donor chamber cap and gasket were soaked in 5 ml of methanol/water (85:15) solution for 30 min. The skin was then removed from the diffusion cell, cut into small pieces, and extracted with 4×1 ml of methanol/water (85:15) solution. Blank skin samples were used as the control. The samples collected from the receptor chamber, the rinses, and skin extraction were centrifuged (3000×g for 15 min), and OXB content in the supernatant was determined using HPLC. Statistical analysis was performed using the Student's unpaired t-test and p<0.05 was considered significant.

### Example 8: HPLC

The HPLC assay was conducted using a Shimadzu HPLC system (Shimadzu Scientific Instruments, Inc., Addison, IL) with an LC-20 AT pump, an SPD-20A UV-Vis detector, a SL-20A autoinjector, and a Microsorb-MV100-5 C18 column (150×4.6 mm, Agilent Technologies, Santa Clara, CA). The mobile phase was methanol and water at a ratio of 85:15. The flow rate was 0.8 ml/min. The detection wavelength for OXB was 294 nm (absorption maximum of OXB) and the detection limit was 10 ng/ml. A calibration curve was constructed using OXB standards at concentrations between 10 ng/ml and 120 μg/ml and linearity was observed in this range. The concentration of OXB in the samples was calculated based on the calibration curve. To examine whether the recovery was related to the extraction method using the mobile phase, a control recovery study was performed in which skin in a vial was dosed with OXB with and without RsC1. The recovery of OXB was 83.6% and that of OXB+RsC1 was 137.4%. In the latter case, the recovery was higher than 100% due to RsC1 interference in the OXB assay, as RsC1 also absorbed at 294 nm and had a similar retention time in the HPLC chromatogram. Therefore, the experimental recovery of OXB in the permeation study was corrected using this information. To determine the concentration of RsC1 and confirm the absence of RsC1 interference in the OXB assay for the receptor chamber samples, calibration curves were constructed at 285 and 325 nm for both OXB and RsC1 using standards of 0-12.5 μg/ml. The calibration curves showed linearity in this concentration range, with RsC1 contributing to the absorbance at 285 nm but that at 325 nm being predominantly due to OXB (AUC vs. concentration slopes of 1.2×106 and 5.4×105 for OXB and RsC1, respectively, at 285 nm, and 8.3×105 and 2.8×102 for OXB and RsC1, respectively, at 325 nm). The concentration of RsC1 in the receptor chamber at 24 h was calculated from comparing the concentration difference between the concentration of OXB at 325 nm (at this wavelength the absorption of RsC1 was negligible) and the concentration of OXB at 285 nm (at this wavelength both OXB and RsC1 had absorption).

Our results show that OXB and RsC1 formed a 1:1 host-guest complex and that complexation dramatically decreased the skin permeation of OXB. Owing to the noncovalent interactions involved in host-guest complexation, this approach can be used to improve the chemical properties of the guest without altering its chemical composition.

TABLE 2
Control recovery (%) of dosing in an empty vial

| | Recovery | | Average |
|---|---|---|---|
| OXB-1 | 98.2 | OXB | 98.3 |
| OXB-2 | 98.1 | | |
| OXB-3 | 98.6 | | |
| OXB + RsC1-1 | 140.7 | OXB + RsC1 | 135.6 |
| OXB + RsC1-2 | 126.2 | | |
| OXB + RsC1-3 | 140.0 | | |
| RsC1-1 | 38.6 | RsC1 | 37.0 |
| RsC1-2 | 34.8 | | |
| RsC1-3 | 36.3 | | |

Table 2: Control recovery (%) of dosing in an empty vial. Group1: OXB; Group 2: OXB+RsC1; Group 3: RsC1. All the compounds were dissolved in ethanol, n=3. Solutions of 0.3% OXB and OXB+RsC1 in ethanol were applied in the empty vials. The vials were left open for 24 h. The vials were then rinsed using 4 ml of 85:15 methanol:water solvent. The amounts of OXB were determined by HPLC at 294 nm. The average recovery from Group 1 was 98.3%. For the OXB+RsC1 complex (Group 2), it was found that the recovery was significantly higher than 100% (average of 135.6%). The reason for the higher than 100% recovery was the interference of RsC1 in the HPLC assay. RsC1 had similar HPLC retention time as OXB and UV absorption at 294 nm. The absorbance corresponding to RsC1 at 294 nm was 37%, resulting in the higher than 100% recovery (98% from OXB+37% from RsC1=135% total).

TABLE 3
Control recovery (%) of dosing on skin in a vial group

| Skin name | OXB group | OXB + RsC1 group |
|---|---|---|
| CC | 86.9 | 130.1 |
| RT | 83.7 | 138.6 |
| SL | 82.3 | 149.3 |
| SW | 81.7 | 131.7 |
| Average | 83.6 | 137.4 |

Table 3: Control recovery (%) of dosing on skin in a vial. Group 1: OXB; Group 2: OXB+RsC1. Split thickness skin was cut into 1.5 cm×1.5 cm pieces and thawed in a petri dish with PBS at room temperature for 2 h. The skin was patted dry with Kimwipe and then put in a vial with the stratum corneum side facing up (n=4 for each group). Solutions of 0.3% OXB and OXB+RsC1 in ethanol were applied directly on the skin surface in the vials. The skin was left in the open vials for 24 h and then rinsed using 1 ml of 85:15 methanol:water solvent. The skin was cut into very small pieces, and then 1 ml of 85:15 methanol:water solvent was added to the vials for extraction. The extraction was performed in the same vials to avoid mass loss. The vials were sonicated for 2 min and then centrifuged. This procedure was repeated 3 times. After the centrifugation, fresh 85:15 methanol:water solvent was added to the skin residue to repeat the extraction overnight. The amount of OXB extracted from the skin was determined by HPLC at 294 nm. This result was used to correct the skin surface rinse, skin cut-up, and donor chamber cap data in the skin permeation recovery study.

TABLE 4
OXB concentrations

| | OXB + RsC1 group | OXB + RsC1 group |
|---|---|---|
| Skin name | at 325 nm | at 285 nm |
| CC | 1.63 | 1.66 |
| RT | 0.95 | 0.94 |
| SL | 2.25 | 2.26 |

Table 4: OXB concentrations (μg/ml) determined at 285 and 325 nm in the HPLC assay using the OXB calibration curves at these two wavelengths, respectively, for the OXB+RsC1 group in the skin permeation study. The UV absorption peak of RsC1 was at 285 nm, and its absorbance at 325 nm was negligible compared to that of OXB (the extinction coefficient of OXB was at least 200× higher than that of RsC1 at 325 nm). HPLC assays at 285 and 325 nm were used to determine the concentration of RsC1 in the receptor chamber after 24 h in the skin permeation study. The calibration curve of OXB at 325 nm was used to calculate the concentration of OXB in the receptor chamber as the absorbance of RsC1 at this wavelength was negligible. Using the calibration curve of OXB at 285 nm, the concentration of OXB was also calculated. Student's unpaired t-test was performed, and there was no significant difference between the concentration of OXB at 325 nm (at this wavelength, the absorbance of RsC1 was negligible) and the concentration of OXB at 285 nm (at this wavelength, both OXB and RsC1 contributed to the absorbance), p=0.22. The lack of difference between the OXB concentrations determined at 285 and 325 nm indicates no contribution of RsC1 to the absorbance in the receptor chamber samples. The maximum concentration of RsC1 in the receptor chamber samples was also estimated by the difference between the absorbance measured at 280 nm and the absorbance at 280 nm calculated using the OXB concentration determined at 325 nm; the OXB absorbance at 280 nm was determined by OXB concentration obtained at 325 nm and the calibration curve at 280 nm. The maximum RsC1 concentration from this estimation was 0.06 μg/ml. This analysis suggests no significant permeation of RsC1 through the skin under the conditions studied.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

1. (canceled)

2. (canceled)

3. (canceled)

4. (canceled)

5. (canceled)

6. (canceled)

7. A method of protecting a subject's skin from sun damage comprising applying a therapeutically effective amount of a composition comprising a cocrystal complex of oxybenzone with C-methylresorcin[4]arene (RsC1) to the skin of the subject.

8. A method of protecting a subject's skin from sun damage comprising applying a therapeutically effective amount a sunscreen formulation comprising a cocrystal complex of oxybenzone with C-methylresorcin[4]arene (RsC1) to the skin of a subject in need of such treatment; wherein the cocrystal complex comprises from about 2 to about 10% by weight of the formulation.

9. The method of claim 8 wherein the cocrystal complex comprises from about 4 to about 8% by weight of the formulation.

10. The method of claim 8 wherein the sunscreen formulation further comprises a cosmetically acceptable carrier.

11. The method of claim 10 wherein said carrier comprises one or more carriers selected from the group consisting of preservatives, emollients, emulsifying agents, surfactants, moisturizers, gelling agents, thickening agents, conditioning agents, film-forming agents, stabilizing agents, anti-oxidants, texturizing agents, gloss agents, mattifying agents, solubilizers, pigments, dyes, and fragrances.
